(19) United States
(12) Patent Application Publication
(10) Pub. No.: US 2024/0077415 A1
(43) Pub. Date: Mar. 7, 2024

(54) TRANSFORMER LIFETIME EVALUATION APPARATUS AND METHOD

(71) Applicant: LS ELECTRIC CO., LTD., Anyang-si, Gyeonggi-do (KR)

(72) Inventor: Jae-Hun YOON, Anyang-si, Gyeonggi-do (KR)

(21) Appl. No.: 18/272,421

(22) PCT Filed: Dec. 22, 2021
(86) PCT No.: PCT/KR2021/019662

§ 371 Date: Jul. 14, 2023

(30) Foreign Application Priority Data
Jan. 15, 2021 (KR) 10-2021-0006152
Apr. 1, 2021 (KR) 10-2021-0042457

(51) Int. Cl. G01N 21/33; G01N 21/41

(52) U.S. Cl. CPC G01N 21/33; G01N 21/41; G01N 2021/1742

(57) ABSTRACT

A transformer lifetime evaluation apparatus comprises a first storage part for storing insulation oil used for insulation of a transformer; a second storage part provided to be separated from the first storage part and storing insulation oil which is not used for the insulation of the transformer; a light-emitting unit for emitting light of a specified wavelength; a light-receiving unit for receiving light emitted from the light-emitting unit; a first optical cable connecting the light-emitting unit and the light-receiving unit, providing a moving path of light emitted from the light-emitting unit and received in the light-receiving unit; a second optical cable connecting the light-emitting unit and the light-receiving unit, providing a moving path of light emitted from the light-emitting unit and received in the light-receiving unit; and a calculation part connected to the light-receiving unit and receiving information about an optical property of methanol from the light-receiving unit.

### CROSS-REFERENCE TO RELATED APPLICATIONS

The present application is a National Stage of International Application No. PCT/KR2021/019662 filed on Dec. 22, 2021, which claims priority to and the benefit of Korean Utility Model Application No. 10-2021-0006152, filed Jan. 15, 2021 and Korean Utility Model Application No. 10-2021-0042457, filed Apr. 1, 2021, the disclosures of which are incorporated herein by reference in its entirety.

### FIELD

The present disclosure relates to a transformer lifetime evaluation apparatus and method, and more particularly, to an oil-immersed transformer lifetime evaluation apparatus and method.

### BACKGROUND

Content described in this section merely provides background information on the present disclosure and does not constitute the related art.

A lifetime of an oil-immersed transformer is related to insulation materials such as insulation oil and insulation paper, and the lifetime of the oil-immersed transformer is determined according to a mechanical lifetime of the insulation paper (cellulose). That is, as the insulation paper is degraded, mechanical strength of the insulation paper is reduced, and the mechanical lifetime of the insulation paper is also reduced. This is because fibrous tissue of the cellulose is weakened by a chemical reaction of cutting long chains of cellulose molecules of the insulation paper.

In this way, since the lifetime of the oil-immersed transformer depends on soundness (a polymerization degree) of the insulation paper, a decrease in the strength of the insulation paper due to the degradation may directly or indirectly cause failure of the transformer. Thus, diagnosis of a state (the polymerization degree) of insulation paper is very important.

A polymerization diagnosis technology includes a method of directly detecting a change in material and an indirect method of detecting a secondary change such as a change in geometry due to degradation and deformation of the material. However, in any method, the diagnosis should be performed during operation (live wire, on-line) without stopping operation of a facility.

In dissolved gas analysis (DGA) that is the related art used for diagnosing an abnormality of a transformer, whether the transformer is abnormal is determined using methane gas that is a dissolved gas generated by the oil-immersed transformer.

However, in the related art, it can only be determined whether the transformer is abnormal, and a relationship between the amount of methane gas and the polymerization degree of the insulation paper is not defined. Thus, the related art cannot be used for evaluating the remaining lifetime of the transformer.

Further, since it is difficult to connect the types of defects in actual transformers and the results measured by DGA to lifetime evaluation of power equipment, a transformer operator should directly inspect the inside of the transformer and visually identify defects when it is determined whether the transformer is abnormal according to a result of the DGA.

Further, a DGA method of analyzing the methane gas according to the related art is not economical because a price of an oil-immersed gas sensor for detecting the dissolved gas is very high.

### SUMMARY

The present disclosure is directed to providing a transformer lifetime evaluation apparatus using optical properties of methanol included in insulation oil.

The present disclosure is also directed to providing a transformer lifetime evaluation method using the transformer lifetime evaluation apparatus.

The present disclosure is also directed to providing a transformer lifetime evaluation method in which a lifetime of a transformer can be evaluated without directly collecting the methanol through a relational expression between the methanol included in the insulation oil and a polymerization degree of insulation paper.

The present disclosure is also directed to providing a transformer lifetime evaluation method in which the lifetime of the transformer is precisely evaluated by calculating the optical properties of the methanol through an optical sensor used as a light source.

The present disclosure is also directed to providing a transformer lifetime evaluation method in which, using the optical sensor that is inexpensive and is easily installed and operated, the lifetime of the transformer is efficiently evaluated, and costs are reduced.

The purposes of the present disclosure are not limited to the purposes described above, and other purposes and advantages of the present disclosure that are not described may be understood from the following description and may be more clearly understood by embodiments of the present disclosure. Further, it may be easily identified that the purposes and advantages of the present disclosure may be implemented by units and combinations thereof described in the appended claims.

A transformer lifetime evaluation apparatus using optical properties of methanol included in insulation oil is disclosed.

One aspect of the present disclosure provides a transformer lifetime evaluation apparatus including a first storage part in which insulation oil used for insulating a transformer is stored, a second storage part which is provided separately from the first storage part and in which insulation oil not used for insulating the transformer is stored, a light emitting unit that emits a light beam having a specific wavelength, a light receiving unit that receives the light beam emitted from the light emitting unit, a first optical cable that connects the light emitting unit and the light receiving unit, provides a movement path for the light beam emitted from the light emitting unit and received by the light receiving unit, and is disposed to pass through the first storage part, a second optical cable that connects the light emitting unit and the light receiving unit, provides a movement path for the light beam emitted from the light emitting unit and received by the light receiving unit, and is disposed to pass through the second storage part, and a calculation unit that is connected to the light receiving unit and receives information on the optical properties of the methanol from the light receiving unit.

The light receiving unit may transmit, to the calculation unit, the information on the optical properties of the methanol included in the insulation oil stored in the first storage part and the second storage part.

The optical properties may include at least one of absorptivity, reflectance, or a refractive index.

The calculation unit may include a methanol content analysis unit that calculates a methanol content included in the insulation oil of the transformer based on the light beam having the specific wavelength, a polymerization degree calculation unit that calculates a polymerization degree of insulation paper provided in the transformer based on the calculated methanol content, and a lifetime evaluation unit that evaluates a lifetime of the transformer based on the calculated polymerization degree.

The methanol content analysis unit may calculate the methanol content included in the insulation oil of the transformer based on an absorbance of the methanol transmitted from the light receiving unit.

The specific wavelength may be in a band in a range of 317 nm to 328 nm.

The calculation unit may compare the optical properties of the methanol stored in the first storage part and the second storage part and determine that the transformer is unusable when a difference value between the optical properties exceeds a set range.

A transformer lifetime evaluation method using the transformer lifetime evaluation apparatus is disclosed.

Another aspect of the present disclosure provides a transformer lifetime evaluation method including emitting, by the light emitting unit, the light beam having the specific wavelength to the insulation oil stored in the first storage part and the second storage part, transmitting, by the light receiving unit, the information on the optical properties of the methanol stored in the first storage part and the second storage part to the calculation unit, and comparing, by the calculation unit, the optical properties of the methanol stored in the first storage part and the second storage part.

In the comparing of, by the calculation unit, the optical properties of the methanol stored in the first storage part and the second storage part , when a difference value between the optical properties exceeds a set range, the calculation unit may determine that the transformer is unusable.

The transformer lifetime evaluation method may further include calculating, by the calculation unit, a content of the methanol included in the insulation oil stored in the first storage part, calculating, by the calculation unit, a polymerization degree of insulation paper provided in the transformer based on the methanol content of the first storage part, and evaluating, by the calculation unit, a lifetime of the transformer based on the calculated polymerization degree.

In the evaluating of, by the calculation unit, the lifetime of the transformer based on the calculated polymerization degree, when the calculated polymerization degree is smaller than or equal to a set lifetime point limit, it may be determined that the transformer is unusable.

Another aspect of the present disclosure provides a sensor module including a first storage part in which insulation oil used for insulating a transformer is stored, a second storage part which is provided separately from the first storage part and in which insulation oil not used for insulating the transformer is stored, a light emitting unit that emits a light beam having a specific wavelength, a light receiving unit that receives the light beam emitted from the light emitting unit, a first optical cable that connects the light emitting unit and the light receiving unit, provides a movement path for the light beam emitted from the light emitting unit and received by the light receiving unit, and is disposed to pass through the first storage part, and a second optical cable that connects the light emitting unit and the light receiving unit, provides a movement path for the light beam emitted from the light emitting unit and received by the light receiving unit, and is disposed to pass through the second storage part.

Still another aspect of the present disclosure provides a transformer lifetime evaluation method including providing a light beam having a predetermined specific wavelength to insulation oil or receiving the light beam having the predetermined specific wavelength, acquiring a methanol content included in the insulation oil of the transformer using the light beam having the specific wavelength, calculating a polymerization degree through the acquired methanol content, and evaluating a lifetime of the transformer based on the calculated polymerization degree.

The optical properties may include absorptivity, reflectance, and a refractive index.

The calculating of the methanol content included in the insulation oil of the transformer based on the provided light beam may include acquiring an absorbance according to the optical properties of the methanol based on the provided light beam, and calculating the methanol content included in the insulation oil of the transformer based on the acquired absorption.

The predetermined specific wavelength may be in a wavelength band of a second slope section.

The calculating of the polymerization degree through the acquired methanol content may be performed through [Equation 1].

$$DP = exp [ \frac{a - ln ( {MeOH}_{ppm} )}{b} ] \qquad [ Equation 1 ]$$

(DP denotes the polymerization degree, MeOH denotes the methanol content, a denotes a first reference value, and b denotes a second reference value)

The evaluating of the lifetime of the transformer based on the calculated polymerization degree may include determining that the transformer is unusable when the calculated polymerization degree is smaller than or equal to a predetermined lifetime point limit.

The lifetime point limit may correspond to a case in which the polymerization degree is 400.

Yet another aspect of the present disclosure provides a transformer lifetime evaluation apparatus including a sensor module that provides a light beam having a predetermined specific wavelength to insulation oil or receives the light beam having the predetermined specific wavelength, a methanol content analysis unit that acquires a methanol content included in the on the insulation oil of the transformer based on the light beam having the specific wavelength, a polymerization degree calculation unit that calculates a polymerization degree through the acquired methanol content, and a lifetime evaluation unit that evaluates a lifetime of the transformer based on the calculated polymerization degree.

The optical properties may include absorptivity, reflectance, and a refractive index.

The methanol content analysis unit may acquire an absorbance according to the optical properties of the methanol based on the light beam having the specific wavelength and calculates a methanol content included in the insulation oil of the transformer based on the acquired absorbance.

The predetermined specific wavelength may be in a wavelength band of a section having an absorbance of a specific magnitude or more while having a graph distribution at regular intervals.

The wavelength band of the section having the absorbance of the specific magnitude or more while having the graph distribution at regular intervals may be greater than or equal to 317 nm and smaller than or equal to 328 nm.

The polymerization degree calculation unit may calculate the polymerization degree through [Equation 1].

$$DP = exp [ \frac{a - ln ( {MeOH}_{ppm} )}{b} ] \qquad [ Equation 1 ]$$

(DP denotes the polymerization degree, Me0H denotes the methanol content, a denotes the first reference value, and b denotes the second reference value)

The lifetime evaluation unit may determine that the transformer is unusable when the calculated polymerization degree is smaller than or equal to a predetermined lifetime point limit.

The lifetime point limit may correspond to a case in which the polymerization degree is 400.

### ADVANTAGEOUS EFFECTS

A transformer lifetime evaluation apparatus according to the present disclosure can easily and precisely evaluate a lifetime of a transformer without directly collecting methanol included in insulation oil through a relational expression between the methanol and a polymerization degree of insulation paper.

Further, the transformer lifetime evaluation apparatus according to the present disclosure can compare optical properties of the methanol stored in a first storage part and a second storage part and thus easily determine whether the transformer reaches a limit lifetime without calculating a polymerization degree of the methanol stored in the first storage part.

In a transformer lifetime evaluation method according to the embodiment of the present specification, the lifetime of the transformer can be evaluated without directly collecting the methanol through a relational expression between the methanol included in the insulation oil and the polymerization degree of the insulation paper.

Further, in the transformer lifetime evaluation method according to the embodiment of the present specification, the lifetime of the transformer can be precisely evaluated by calculating the optical properties of the methanol through the optical sensor that uses a light beam having a specific wavelength as a light source.

Further, in the transformer lifetime evaluation method according to the embodiment of the present specification, the optical sensor that has low cost and is easily installed and operated is used, the lifetime of the transformer can be efficiently evaluated, and thus costs can be reduced.

In addition to the above-described effects, the detailed effects of the present disclosure will be described together while specific details for implementing the disclosure are described below.

### BRIEF DESCRIPTION OF THE DRAWINGS

FIG. 1A is a schematic block diagram illustrating a transformer lifetime evaluation apparatus according to an embodiment.

FIG. 1B is a schematic view illustrating a sensor module provided in the transformer lifetime evaluation apparatus according to the embodiment.

FIG. 2 is a graph depicting absorbance of methanol for all wavelengths according to the embodiment.

FIG. 3 is an enlarged view of a graph in a specific wavelength area in the graph of FIG. 2.

FIG. 4 is a graph depicting a polymerization degree and a change in methanol content of the insulation oil according to a degradation time according to the embodiment.

FIG. 5 is a flowchart illustrating a transformer lifetime evaluation method according to the embodiment.

FIG. 6 is a block diagram of the transformer lifetime evaluation apparatus and a transformer according to the embodiment of the present specification.

FIG. 7 is a graph depicting absorbance of methanol for all wavelengths in the embodiment of the present specification.

FIG. 8 is an enlarged view of a graph in a specific wavelength area in the graph of FIG. 7.

FIG. 9 is a graph depicting the polymerization degree and the change in the methanol content of the insulation oil according to the degradation time in the embodiment of the present specification.

FIG. 10 is a flowchart of the transformer lifetime evaluation method according to the embodiment of the present specification.

### DETAILED DESCRIPTION

The above-described purposes, features, and advantages will be described in detail with reference to the accompanying drawings, and accordingly, those skilled in the art to which the present disclosure pertains may easily implement the technical spirit of the present disclosure. In the description of the present disclosure, when it is determined that detailed description of widely known technologies related to the present disclosure may make the subject matter of the present disclosure unclear, the detailed description will be omitted. Hereinafter, embodiments of the present disclosure will be described in detail with reference to the accompanying drawings. In the drawings, the same reference numerals are used to indicate the same or similar components.

Although “first,” “second,” and the like are used to describe various components, it is apparent that these components are not limited by these terms. These terms are only used to distinguish one component from another component, and it is apparent that a first component may be a second component unless particularly otherwise stated.

Throughout the specification, unless particularly otherwise stated, each component may be singular or plural.

Singular expressions used herein include plural expressions unless clearly otherwise indicated in the context. In the present application, terms such as “configuring” or “including” should not be interpreted as necessarily including all of various components or various steps described in the specification and should be interpreted as not including some components or some steps thereof or further including additional components or additional steps.

Throughout the specification, when “A and/or B” is used, this means A, B or A and B unless otherwise stated, and when “C to D” is used, this means that a value is greater than or equal to C and less than or equal to D unless otherwise stated.

### First Embodiment

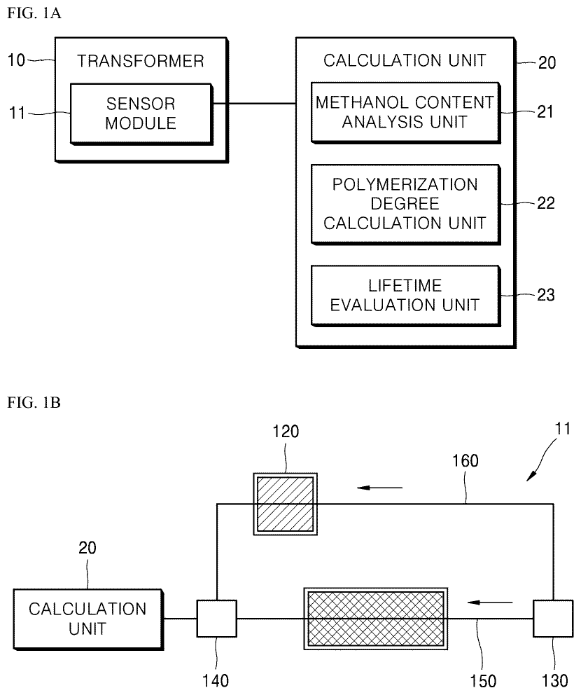

FIG. 1A is a schematic block diagram illustrating a lifetime evaluation apparatus of a transformer 10 according to an embodiment. FIG. 1B is a schematic view illustrating a sensor module 11 provided in the lifetime evaluation apparatus for the transformer 10 according to the embodiment. In FIG. 1B, an arrow indicates a traveling direction of a light beam.

The transformer 10 is a device that transfers electric energy between two or more circuits through an inductive electric conductor and increases or decreases a voltage. In the present specification, the transformer 10 may be an oil-immersed transformer 10 in which a coil wound around an iron core inside the transformer 10 is insulated with insulation oil. In the following description, it will be assumed that the transformer 10 is the oil-immersed transformer 10.

The transformer 10 includes the insulation oil and insulation paper. The insulation oil may be accommodated inside the transformer 10, and the insulation paper may be provided to surround the coil.

The insulation oil and the insulation paper are accommodated inside the transformer 10 and perform an electrical insulation function to prevent an electric shock accident from occurring in the transformer 10. For example, the insulation paper may be cellulose insulation paper, and the insulation oil may be mineral oil, synthetic oil, polychlorinated biphenyl (PCB), mixed oil, or an alkyl benzene. However, the present disclosure is not limited thereto.

Meanwhile, the insulation paper is degraded as the number of operating years of the transformer 10 increases. In detail, since a temperature inside the transformer 10 increases as the transformer 10 operates, the insulation paper is subjected to temperature stress.

When the insulation paper is subjected to the temperature stress, methanol is generated as a secondary compound while the insulation paper is decomposed by thermal energy, and the generated methanol is dissolved in the insulation oil inside the transformer 10. Thus, as the number of operating years of the transformer 10 increases, the content of the methanol in the insulation oil of the transformer 10 accumulates and increases.

The lifetime evaluation apparatus according to the embodiment may use optical properties of the methanol included in the insulation oil. That is, as the insulation paper is degraded, the optical properties of the methanol included in the insulation oil of the transformer 10, for example, absorptivity, reflectance, or a refractive index of the methanol, change. In this case, the lifetime evaluation apparatus may measure these changes to evaluate the lifetime of the transformer 10.

The lifetime evaluation apparatus according to the embodiment may include a sensor module 11 and a calculation unit 20 connected to the sensor module 11 to be able to communicate with the sensor module 11. The sensor module 11 may be provided in the transformer 10, and the calculation unit 20 may be provided in the transformer 10 or separately provided outside the transformer 10.

The sensor module 11 may use, for example, an optical sensor, a chemical sensor, or an electrical sensor. However, since the optical sensor is affected less by the surrounding environment, it will be assumed in the description that the sensor module 11 uses an optical sensor.

The sensor module 11 may include a first storage part 110, a second storage part 120, a light emitting unit 130, a light receiving unit 140, a first optical cable 150, and a second optical cable 160.

The first storage part 110 may store the insulation oil used for insulating the transformer 10. The first storage part 110 may be the entire insulation oil storage device of the transformer 10. Since the insulation oil actually used for insulating the transformer 10 is stored in the first storage part 110, the methanol that is a substance generated by degrading the insulation paper may be included in the insulation oil stored in the first storage part 110. This is because the methanol is generated from the insulation paper as the insulation paper is degraded and is dissolved in the insulation oil stored in the first storage part 110.

In the embodiment, a methanol content of the insulation oil stored in the first storage part 110 is obtained, the polymerization degree of the insulation paper is obtained based on the methanol content, and thus a degree of degradation of the insulation paper is determined. Further, the lifetime of the transformer 10 can be evaluated based on the degree of degradation.

The second storage part 120 may be provided separately from the first storage part 110 and may store the insulation oil that is not used for insulating the transformer 10. To prevent the insulation oil stored in the second storage part 120 from being mixed with the insulation oil of the first storage part 110, the second storage part 120 may be sealed in a state in which the insulation oil not used for the insulation is stored therein.

The second storage part 120 may be disposed in a space provided at an appropriate position inside the transformer 10. For example, to save a space, the first storage part 110 may be accommodated inside the second storage part 120.

The insulation oil for comparison with the insulation oil stored in the first storage part 110 may be accommodated in the second storage part 120. Thus, the second storage part 120 may have a small volume and store a small amount of the insulation oil as compared to the first storage part 110. Since the second storage part 120 is separated from the first storage part 110, the insulation oil in the second storage part 120 does not include the methanol generated from the insulation paper and stored in the first storage part 110.

The sensor module 11 may include the light emitting unit 130 and the light receiving unit 140. That is, the sensor module 11 provides a light beam to the first storage part 110 and the second storage part 120 through the light emitting unit 130 and receives the provided light beam through the light receiving unit 140.

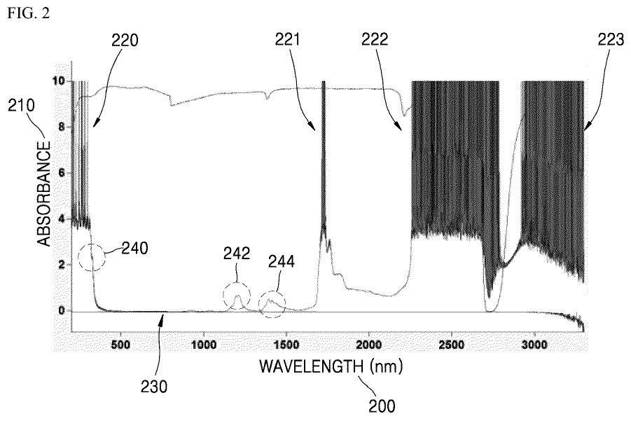

The light emitting unit 130 may emit a light beam having a specific wavelength. The specific wavelength, which is in a wavelength band in which the optical properties of the methanol are clearly distinguished, may be, for example, greater than or equal to 317 nm and smaller than or equal to 328 nm. The specific wavelength will be described below in detail with reference to FIGS. 2 to 4.

The light receiving unit 140 may receive the light beam emitted by the light emitting unit 130. The light emitting unit 130 may emit the light beam having a predetermined specific wavelength to the insulation oil and receive optical properties of the insulation oil. In detail, the light receiving unit 140 may receive the optical properties of the methanol included in the insulation oil stored in the first storage part 110 and the second storage part 120.

The first optical cable 150 may be disposed to connect the light emitting unit 130 and the light receiving unit 140, provide a movement path for the light beam emitted from the light emitting unit 130 and received by the light receiving unit 140, and pass through the first storage part 110.

The light beam passing through the first optical cable 150 may be emitted to the methanol included in the insulation oil stored in the first storage part 110. Thus, the light receiving unit 140 may receive the optical properties of the methanol in the first storage part 110 through the first optical cable 150.

The second optical cable 160 may be disposed to connect the light emitting unit 130 and the light receiving unit 140, provide the movement path for the light beam emitted from the light emitting unit 130 and received by the light receiving unit 140, and pass through the second storage part 120.

The light beam passing through the second optical cable 160 may be emitted to the insulation oil stored in the second storage part 120. Since the methanol does not flow into the second storage part 120 from the insulation paper, the light receiving unit 140 may receive, through the second optical cable 160, the optical properties of the insulation oil that does not contain the methanol. In the following description, the optical properties of the methanol may include the optical properties of the insulation oil in a state in which the methanol is not included.

The methanol has different optical properties depending on the wavelength of the light beam. Here, the optical properties may include at least one of the absorptivity, the reflectance, or the refractive index. In detail, the absorptivity is a degree to which the methanol absorbs the light beam, the reflectance is a degree to which the methanol reflects the light beam, and the refractive index is a degree to which the light beam is bent at an interface when the light beam passes through the methanol. In the same wavelength, the optical properties of the methanol, that is, the absorptivity, the reflectance, and the refractive index, are all similar properties.

The calculation unit 20 may be connected to the light receiving unit 140 and may receive information on the optical properties of the methanol from the light receiving unit 140. In this case, the light receiving unit 140 may transmit, to the calculation unit 20, the information on the optical properties of the methanol included in the insulation oil stored in the first storage part 110 and the second storage part 120.

The calculation unit 20 may include a methanol content analysis unit 21, a polymerization degree calculation unit 22, and a lifetime evaluation unit 23.

The methanol content analysis unit 21 may calculate, based on the light beam having a specific wavelength, the methanol content included in the insulation oil of the transformer 10. The methanol content analysis unit 21 may calculate the methanol content included in the insulation oil of the transformer 10 based on the absorbance of the methanol transmitted from the light receiving unit 140.

In detail, the methanol content analysis unit 21 may acquire the absorbance according to the optical properties of the methanol based on the light beam provided by the sensor module 11 and calculate the methanol content included in the insulation oil of the transformer 10 based on the acquired absorbance.

The absorbance is a log value of a ratio of emitted radiation to transmitted radiation when the methanol is illuminated. In other words, the absorbance indicates a degree to which the methanol absorbs a light beam having a specific wavelength and is proportional to absorptivity, which is an optical property of methanol. Thus, as the amount of the light beam absorbed by the methanol increases, the absorbance increases, and as the amount of the absorbed light beam decreases, the absorbance decreases.

Further, when the absorbance is high, the methanol content included in the insulation oil of the transformer 10 is large, and when the absorbance is low, the methanol content included in the insulation oil of the transformer 10 is low. Accordingly, the methanol content analysis unit 21 calculates, based on the acquired absorbance, the methanol content included in the insulation oil of the transformer 10.

The polymerization degree calculation unit 22 may calculate the polymerization degree of the insulation paper provided in the transformer 10 based on the calculated methanol content. Here, the polymerization degree is an extent to which soundness of the insulation paper, that is, the degradation of the insulation paper, has progressed. Thus, as the degradation progresses, the polymerization degree decreases, and a mechanical lifetime of the insulation paper is reduced. A method of calculating the polymerization degree will be described below in detail.

The lifetime evaluation unit 23 may evaluate the lifetime of the transformer 10 based on the calculated polymerization degree. In detail, the lifetime evaluation unit 23 may preset a lifetime point limit of the transformer 10. The lifetime point limit corresponds to a case in which the calculated polymerization degree is, for example, 400. The lifetime point limit will be described below in detail.

The lifetime evaluation unit 23 may compare the calculated polymerization degree with the preset lifetime point limit and determine that the transformer 10 is unusable when the calculated polymerization degree is smaller than or equal to the preset lifetime point limit.

Further, the lifetime evaluation unit 23 may compare the calculated polymerization degree with the preset lifetime point limit and evaluate the residual lifetime of the transformer 10 according to a magnitude of a difference value between the preset lifetime point limit and the calculated polymerization degree when the calculated polymerization degree is greater than the lifetime point limit.

The calculation unit 20 may compare the optical properties of the methanol stored in the first storage part 110 and the second storage part 120 and determine that the transformer 10 is unusable when a difference value between the optical properties exceeds a set range.

In this case, since the methanol generated from the insulation paper does not flow into the second storage part 120, the calculation unit 20 may receive the optical properties, that is, second optical properties, of the insulation oil in the second storage part 120, in which the methanol is not included, through the light receiving unit 140. Further, the calculation unit 20 may receive, from the light receiving unit 140, the optical properties, that is, the second optical properties, of the methanol stored in the first storage part 110.

As the operating years of the transformer 10 increase, the insulation paper is degraded, and thus first optical properties may change. However, since the methanol generated from the degradation of the insulation paper does not flow into the second storage part 120, the second optical properties do not change or change very little.

Thus, the calculation unit 20 may compare the first optical properties and the second optical properties and determine that the transformer 10 is unusable when a difference value therebetween exceeds a set range. In this case, the set range for the difference value between the first optical properties and the second optical properties may be appropriately selected based on the lifetime point limit.

For example, difference values between the absorptivity, the reflectance, or the refractive index of the insulation oil at the lifetime point limit and the absorptivity, the reflectance, or the refractive index of the insulation oil that is not yet used for insulation may be selected as set values. The set values may be determined based on a lifetime point limit that is already derived from a transformer 10 other than the current transformer 10.

In this case, when at least one of the difference values between the absorptivities, the reflectances, or the refractive indexes of the first optical properties and the second optical properties deviates from the set value, the calculation unit 20 may determine that the current transformer 10 is unusable.

In an embodiment, as the optical properties of the methanol stored in the first storage part 110 and the second storage part 120 are compared, whether the transformer 10 reaches the lifetime point limit can be determined without calculating the polymerization degree of the methanol stored in the first storage part 110.

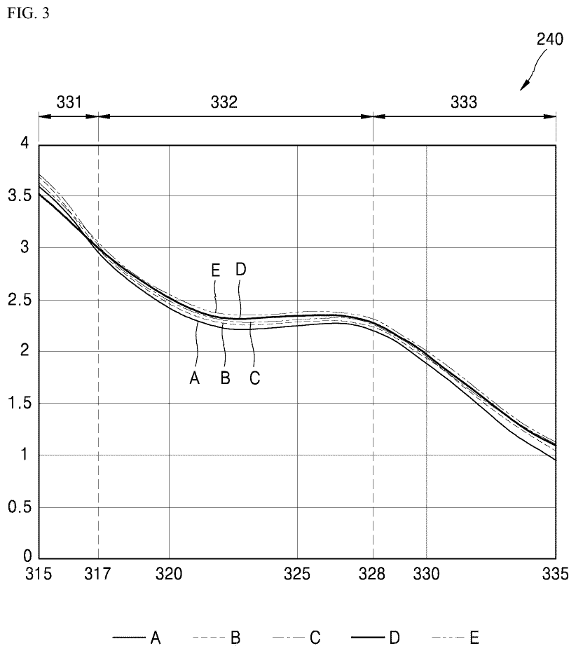

FIG. 2 is a graph depicting absorbance of methanol for all wavelengths according to the embodiment. FIG. 3 is an enlarged view of a graph in a specific wavelength area in the graph of FIG. 2.

Referring to FIG. 2, in the graph, a horizontal axis denotes a wavelength (nm) 200 of a light beam, and a vertical axis denotes an absorbance 210. The wavelength 200 of the light beam is classified into different areas according to a length thereof. The different areas of the light beam may be classified into, for example, gamma rays, X-rays, ultraviolet rays (100 nm to 380 nm), visible rays (380 nm to 780 nm), infrared rays (780 nm to 1000 nm), ultrasonic waves, radio waves, and the like, and the respective areas have different properties.

In this case, as illustrated in FIG. 2, since noises 220, 221, 222, and 223 occur in specific sections, it is impossible to measure the absorbance and it is difficult to identify the content of the methanol in a section 230 in which a slope of the graph is very gentle.

As a result, the content of the methanol can be measured in a first section 240, a second section 242, and a third section 244 in which no noise occurs and the slope is present. However, as the absorbance becomes lower, it is difficult to distinguish the noises when the content of the methanol is measured, a precise technology is required, and accordingly, much cost is consumed.

Referring back to FIG. 2, since the first section 240 has the absorbance that is greater than those of the second section 242 and the third section 244, the first section 240 is advantageous in measuring the methanol content. That is, the first section 240 has a high absorbance of 2 or more for a wavelength in an ultraviolet area, while the second section 242 and the third section 244 have low absorbances of 1 or less for wavelengths other than the ultraviolet area. Thus, when the methanol content analysis unit 21 calculates the content of the methanol in the first section 240, measurement costs can be reduced and economic efficiency can be improved.

Meanwhile, the sensor module 11 may irradiate the insulation oil with a light beam having a predetermined specific wavelength. In detail, referring to FIG. 3 in which the first section 240 is enlarged, a curved graph varies depending on the content of the methanol included in the insulation oil.

That is, graph E denotes a graph of pure insulation oil in which the methanol is not present (degradation of the insulation paper has not progressed), and graph A to graph D denote graphs of the insulation oil in which the methanol is included (degradation of the insulation paper has progressed).

In detail, the content of the methanol increases from graph D to graph A. For example, graph D may indicate the content of the methanol of 3.35 ppm, graph C may indicate the content of the methanol of 4.4 ppm, graph B may indicate the content of the methanol of 32.7 ppm, and graph A may indicate the content of the methanol of 135 ppm.

Further, as the methanol content increases, the absorptivity decreases, and thus when the absorbance that is higher than that of graph E is measured, it is determined that the noise occurs.

Meanwhile, the first section 240 may be divided into a first slope section 331, a second slope section 332, and a third slope section 333. The first slope section 331, the second slope section 332, and the third slope section 333 have different slopes and thus may be classified according to a slope angle.

Here, in the first slope section 331, distributions of graph A to graph E are not uniform, a graph deviating from graph E is present, and thus the noise occurs. Thus, it is difficult to calculate the methanol content according to the absorbance and accurately evaluate the lifetime of the transformer 10.

Further, the third slope section 333 not only has a graph distribution having irregular intervals but also has an absorbance of 2 or less. Thus, it is difficult to precisely measure the methanol content.

However, the second slope section 332 has an absorbance of 2 or more while having a graph distribution at regular intervals. Thus, the methanol content according to a difference in the absorbance can be precisely calculated in the second slope section 332.

A wavelength band of the second slope section 332 may be in a range of 317 nm to 328 mm. Thus, a specific wavelength of the light beam emitted from the light emitting unit 130 of the sensor module 11 may be a wavelength in a band in a range of 317 nm to 328 nm.

However, the second slope section 332 is not limited thereto and may include all specific wavelength areas having an absorbance of 2 or more while having a graph distribution at regular intervals. The methanol content analysis unit 21 may analyze the absorbance of the second slope section 332 to calculate the methanol content.

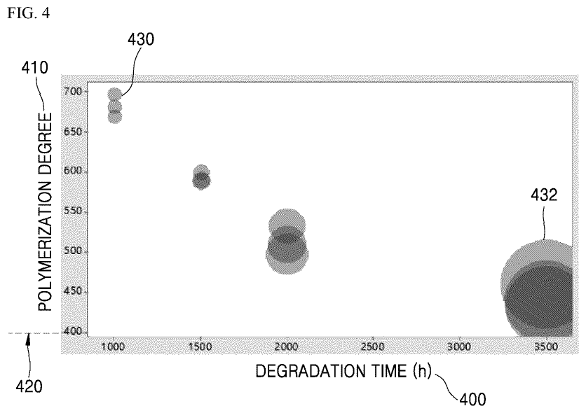

FIG. 4 is a graph depicting a polymerization degree and a change in methanol content of the insulation oil according to a degradation time according to the embodiment.

Referring to the drawing, a horizontal axis denotes a degradation time (h) 400, a vertical axis denotes a polymerization degree 410, and a circle marked in the drawing denotes the methanol content included in the insulation oil. As the degradation time 400 increases, the methanol content of the insulation oil increases due to the degradation of the insulation paper, and thus a diameter of the circle increases.

For example, a diameter 432 of the circle when the degradation time is 3500 h is greater than a diameter 430 of the circle when the degradation time is 1000 h. That is, the methanol content included in the insulation oil is proportional to the degradation time 400.

Meanwhile, the polymerization degree calculation unit 22 may calculate the polymerization degree 410 from the methanol content through [Equation 1].

$$DP = exp [ \frac{a - ln ( {MeOH}_{ppm} )}{b} ] \qquad [ Equation 1 ]$$

(Here, DP denotes the polymerization degree, MeOH denotes the methanol content, a denotes a first reference value, and b denotes a second reference value)

Here, the first reference value may be any value between 56.55 that is a lower limit of the first reference value and 73.5 that is an upper limit of the first reference value, and the second reference value may be any value between 8.5 that is a lower limit of the second reference value and 11.15 that is an upper limit of the second reference value.

Referring back to FIG. 4, the polymerization degree according to the methanol content, which is calculated through [Equation 1], can be identified. When the diameter 432 of the circle is large, the polymerization degree is 650 to 700, which is greater than 400 to 500 that is the polymerization degree when the diameter 430 of the circle is small. That is, as the methanol content increases, the polymerization degree decreases.

In other words, since the polymerization degree of the methanol included in the insulation oil decreases as the degradation of the insulation paper progresses, the lifetime evaluation unit 23 can evaluate the lifetime of the insulation paper, that is, the lifetime of the transformer 10, through the calculated polymerization degree.

In an embodiment, the lifetime evaluation unit 23 may preset the lifetime point limit. The lifetime point limit may be, for example, a polymerization degree of 400 that is a minimum polymerization degree at which it is determined that the transformer 10 is unusable (indicated by reference numeral 420). Thus, the lifetime evaluation unit 23 may evaluate the lifetime of the transformer 10 based on the polymerization degree of 400 and evaluate that the lifetime of the transformer 10 is over and thus the transformer 10 is no longer usable when the calculated polymerization degree is 400 or less.

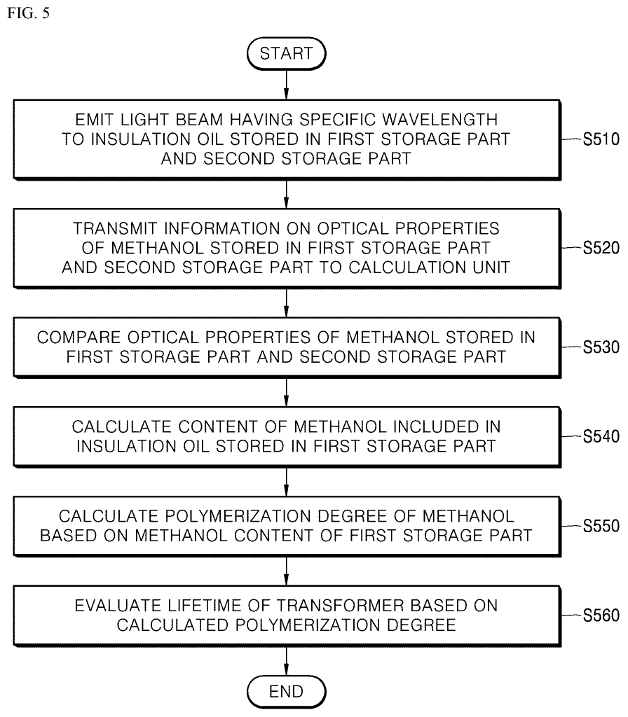

FIG. 5 is a flowchart illustrating a lifetime evaluation method for the transformer 10 according to the embodiment. The lifetime evaluation method for the transformer 10 according to the embodiment may be performed using the above-described lifetime evaluation apparatus for the transformer 10.

The light emitting unit 130 may emit a light beam having a specific wavelength to the insulation oil stored in the first storage part 110 and the second storage part 122 (S510). The light beam emitted from the light emitting unit 130 may pass through the first storage part 110 and the second storage part 120. The light receiving unit 140 may receive the optical properties of the methanol stored in the first storage part 110 and the second storage part 120.

The light receiving unit 140 may transmit, to the calculation unit 20, the information on the optical properties of the methanol stored in the first storage part 110 and the second storage part 120 (S520).

The calculation unit 20 may compare the optical properties of the methanol stored in the first storage part 110 and the second storage part 122 (S530). In operation S530, the calculation unit 20 may determine that the transformer 10 is unusable when a difference value between the optical properties exceeds a set range.

The calculation unit 20 may calculate the content of the methanol included in the insulation oil stored in the first storage part 110 (S540).

The calculation unit 20 may calculate the polymerization degree of the insulation paper provided in the transformer 10 based on the methanol content of the first storage part 110 (S550).

The calculation unit 20 may evaluate the lifetime of the transformer 10 based on the calculated polymerization degree (S560). In operation S560, when the calculated polymerization degree is less than or equal to the set lifetime point limit, the calculation unit 20 may determine that the transformer 10 is unusable. In this case, as described above, the set lifetime point limit may correspond to a case in which the calculated polymerization degree is, for example, 400.

### Second Embodiment

FIG. 6 is a block diagram of the transformer lifetime evaluation apparatus and a transformer according to the embodiment of the present specification.

A transformer 1600 is a device that transfers electric energy between two or more circuits through an inductive electric conductor and increases or decreases a voltage. In the present specification, the transformer 1600 may be an oil-immersed transformer in which a coil wound around an iron core inside the transformer 1600 is insulated with the insulation oil. In the following description, it will be assumed that the transformer 1600 is an oil-immersed transformer.

Referring to the drawing, the transformer 1600 includes insulation oil 1620 and insulation paper 1640.

The insulation oil 1620 and the insulation paper 1640 are accommodated inside the transformer 1600 and perform an electrical insulation function to prevent an electric shock accident occurring in the transformer 1600. For example, the insulation paper 1640 may be cellulose insulation paper, and the insulation oil 1620 may be mineral oil, synthetic oil, polychlorinated biphenyl (PCB), mixed oil, or an alkyl benzene. However, the present disclosure is not limit thereto.

Meanwhile, the insulation paper 1640 is degraded as the number of operating years of the transformer 1600 increases. In detail, as the transformer 1600 operates, a temperature inside the transformer 1600 increases, and thus the insulation paper 1640 is subjected to temperature stress. When the insulation paper 1640 is subjected to the temperature stress, the methanol that is a secondary compound is generated while the insulation paper is decomposed by thermal energy, and the generated methanol is dissolved into the insulation oil 1620 inside the transformer 1600. Thus, as the number of operating years of the transformer 1600 increases, the content of the methanol in the insulation oil 1620 of the transformer 1600 accumulates and increases.

A transformer lifetime evaluation apparatus 1000 according to the embodiment of the present specification, which is an apparatus for evaluating a lifetime of a transformer, may include a sensor module 1100, a methanol content analysis unit 1200, a polymerization degree calculation unit 1300, and a lifetime evaluation unit 1400.

The sensor module 1100 includes a light emitting unit and a light receiving (light measuring) unit. That is, the sensor module 1100 directly provides a light beam to the insulation oil 1620 through the light emitting unit or receives a light beam provided from a separate light emitting element inside the transformer 1600 through the light receiving (light measuring) unit.

The sensor module 1100 may be, for example, an optical sensor, a chemical sensor, or an electrical sensor. However, since the optical sensor is affected less by the surrounding environment, it will be assumed in the description that the sensor module 1100 is an optical sensor.

In the drawing, it is illustrated that the sensor module 1100 is positioned inside the transformer lifetime evaluation apparatus 1000, but in detail, the sensor module 1100 may be disposed inside the transformer 1600 to provide the light beam to the insulation oil 1620 or receive the light beam provided from the separate light emitting element.

Meanwhile, the methanol has different optical properties depending on the wavelength of the light beam. Here, the optical properties are the absorptivity, the reflectance, or the refractive index. In detail, the absorptivity is a degree to which the methanol absorbs the light beam, the reflectance is a degree to which the methanol reflects the light beam, and the refractive index is a degree to which the light beam is bent at an interface when the light beam passes through the methanol. In the same wavelength, the optical properties of the methanol, that is, the absorptivity, the reflectance, and the refractive index, are all similar properties.

The sensor module 1100 may provide a light beam having a predetermined specific wavelength to the insulation oil or receive the light beam having the predetermined specific wavelength. That is, the sensor module 1100 may generate and emit, through the light emitting unit, the light beam having the predetermined specific wavelength or absorb, through the light receiving (light measuring) unit, the light beam having the predetermined specific wavelength from the light beam generated by the separate light emitting element.

The predetermined specific wavelength, which is in a wavelength band in which the optical properties of the methanol are clearly distinguished, may be, for example, greater than or equal to 317 nm and smaller than or equal to 328 nm. The predetermined specific wavelength band will be described below in detail.

The methanol content analysis unit 1200 calculates the content of the methanol included in the insulation oil 1620 of the transformer 1600 based on the provided light beam. In detail, the methanol content analysis unit 1200 may acquire the absorbance according to the optical properties of the methanol based on the light beam provided by the sensor module 1100 and calculate the methanol content included in the insulation oil 1620 of the transformer 1600 based on the acquired absorbance.

The absorbance is a log value of a ratio of emitted radiation to transmitted radiation when the methanol is illuminated. In other words, the absorbance indicates a degree to which the methanol absorbs a light beam having a specific wavelength and is proportional to absorptivity, which is an optical property of methanol. Thus, as the amount of the light beam absorbed by the methanol increases, the absorbance increases, and as the amount of the absorbed light beam decreases, the absorbance decreases.

Further, when the absorbance is high, the methanol content included in the insulation oil 1620 of the transformer 1600 is large, and when the absorbance is low, the methanol content included in the insulation oil 1620 of the transformer 1600 is small. Accordingly, the methanol content analysis unit 1200 calculates, based on the acquired absorbance, the methanol content included in the insulation oil 1620 of the transformer 1600.

The polymerization degree calculation unit 1300 calculates the polymerization degree through the acquired methanol content. Here, the polymerization degree indicates how sound the insulation paper 1640 is, that is, a degree to which degradation of the insulation paper 1640 has progressed. Thus, as the degradation progresses, the polymerization degree decreases, and a mechanical lifetime of the insulation paper 1640 decreases.

In detail, the polymerization degree may be calculated through [Equation 1] related to the methanol content and the polymerization degree of the insulation paper 1640.

$$DP = exp [ \frac{a - ln ( {MeOH}_{ppm} )}{b} ] \qquad [ Equation 1 ]$$

(DP denotes the polymerization degree, Me0H denotes the methanol content, a denotes the first reference value, and b denotes the second reference value)

Here, the first reference value may be any value between 56.55 that is a lower limit of the first reference value and 73.5 that is an upper limit of the first reference value, and the second reference value may be any value between 8.5 that is a lower limit of the second reference value and 11.15 that is an upper limit of the second reference value.

The lifetime evaluation unit 1400 evaluates the lifetime of the transformer 1600 based on the calculated polymerization degree. In detail, the lifetime evaluation unit 1400 may preset a lifetime point limit of the transformer 1600. The lifetime point limit may be, for example, 400.

The lifetime evaluation unit 1400 may compare the calculated polymerization degree with the preset lifetime point limit and determine that the transformer 1600 is unusable when the calculated polymerization degree is smaller than or equal to the preset lifetime point limit.

Further, the lifetime evaluation unit 1400 may compare the calculated polymerization degree with the preset lifetime point limit and evaluate the residual lifetime of the transformer 1600 according to a magnitude of a difference value between the preset lifetime point limit and the calculated polymerization degree when the calculated polymerization degree is greater than the lifetime point limit.

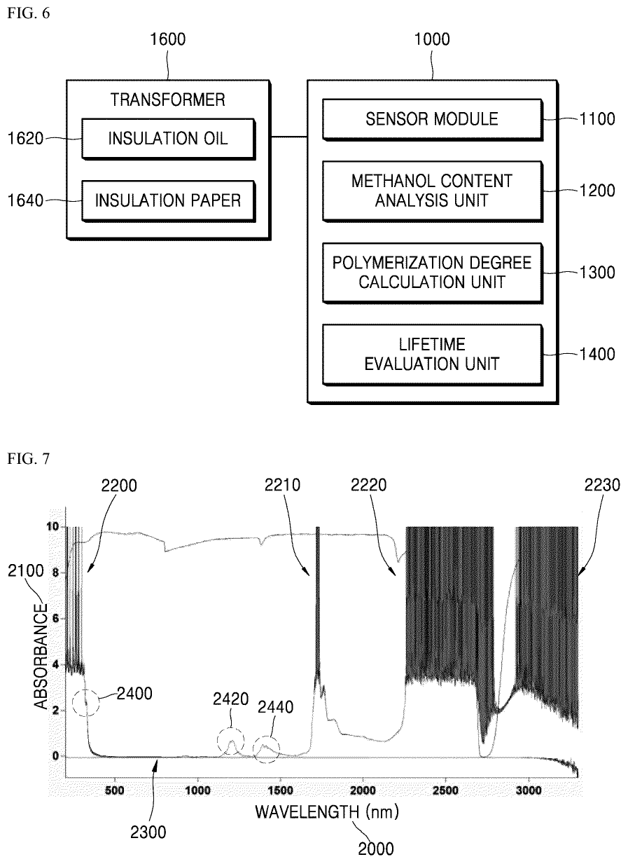

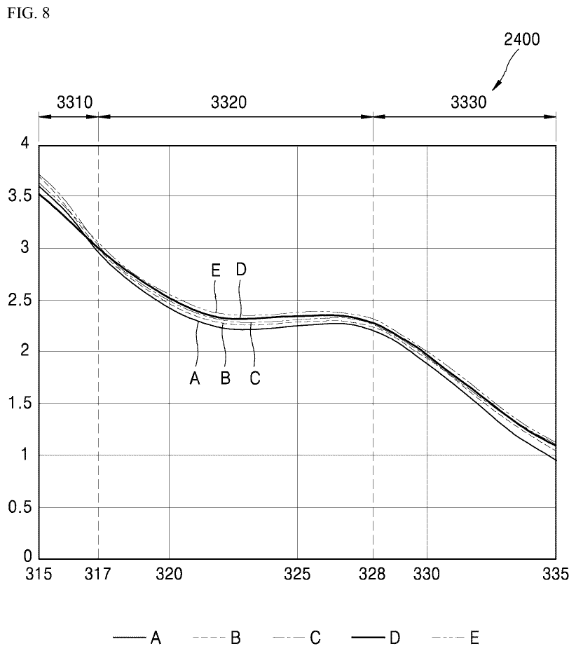

FIG. 7 is a graph depicting absorbance of insulation oil including methanol for all wavelengths in the embodiment of the present specification, and FIG. 8 is an enlarged view of a graph in a specific wavelength area in the graph of FIG. 7. Hereinafter, the graph will be described with reference to FIGS. 7 and 8.

Referring to FIG. 7, in the graph, a horizontal axis denotes a wavelength (nm) 2000 of a light beam, and a vertical axis denotes an absorbance 2100. The wavelength 2000 of the light beam is classified into different areas according to a length thereof. The different areas of the light beam may be classified into, for example, gamma rays, X-rays, ultraviolet rays (100 nm to 380 nm), visible rays (380 nm to 780 nm), infrared rays (780 nm to 1000 nm), ultrasonic waves, radio waves, and the like, and the respective areas have different properties.

In this case, as illustrated in FIG. 7, since noises 2200, 2210, 2220, and 22302230 occur in specific sections, it is impossible to measure the absorbance and it is difficult to identify the content of the methanol in a section 2300 in which a slope of the graph is very gentle.

As a result, the content of the methanol can be measured in a first section 2400, a second section 2420, and a third section 2440 in which no noise occurs and the slope is present. However, as the absorbance becomes lower, it is difficult to distinguish the noises when the content of the methanol is measured, a precise technology is required, and accordingly, much cost is consumed.

Referring back to FIG. 7, since the first section 2400 has the absorbance that is greater than those of the second section 2420 and the third section 2440, the first section 240 is advantageous in measuring the methanol content. That is, the first section 2400 has a high absorbance of 2 or more for a wavelength in an ultraviolet area, while the second section 2420 and the third section 2440 have low absorbances of 1 or less for wavelengths other than the ultraviolet area. Thus, when the methanol content analysis unit 1200 calculates the content of the methanol in the first section 2400, measurement costs can be reduced and economic efficiency can be improved.

Meanwhile, the sensor module 1100 may provide a light beam having a predetermined specific wavelength to the insulation oil. In detail, referring to FIG. 8 in which the first section 2400 is enlarged, a curved graph varies depending on the content of the methanol included in the insulation oil. That is, graph E denotes a graph of pure insulation oil 1620 in which the methanol is not present (degradation of the insulation paper has not progressed), and graph A to graph D denote graphs of the insulation oil in which the methanol is included (degradation of the insulation paper has progressed).

In detail, the content of the methanol increases from graph D to graph A. For example, graph D may indicate the content of the methanol of 3.35 ppm, graph C may indicate the content of the methanol of 4.4 ppm, graph B may indicate the content of the methanol of 32.7 ppm, and graph A may indicate the content of the methanol of 135 ppm.

Further, as the methanol content increases, the absorptivity decreases, and thus when the absorbance that is higher than that of graph E is measured, it is determined that the noise occurs.

Meanwhile, the first section 2400 may be divided into a first slope section 3310, a second slope section 3320, and a third slope section 3330. In detail, the first slope section 3310, the second slope section 3320, and the third slope section 3330 have different slopes and thus may be classified according to a slope angle.

Here, in the first slope section 3310, distributions of graph A to graph E are not uniform, a graph deviating from graph E is present, and thus the noise occurs. Thus, it is difficult to calculate the methanol content according to the absorbance and accurately evaluate the lifetime of the transformer 1600.

Further, the third slope section 3330 not only has a graph distribution having irregular intervals but also has an absorbance of 2 or less. Thus, it is difficult to precisely measure the methanol content.

However, the second slope section 3320 has an absorbance of 2 or more while having a graph distribution at regular intervals. Thus, the methanol content according to a difference in the absorbance can be precisely calculated in the second slope section 3320.

A wavelength band of the second slope section 3320 may be in a range of 317 nm to 328 mm. Thus, in the embodiment of the present specification, the predetermined specific wavelength of the sensor module 1100 may be greater than or equal to 317 nm and smaller than or equal to 328 nm. However, the second slope section 3320 is not limited thereto and may include all specific wavelength areas having an absorbance of 2 or more while having a graph distribution at regular intervals. The methanol content analysis unit 1200 may analyze the absorbance of the second slope section 3320 to calculate the methanol content.

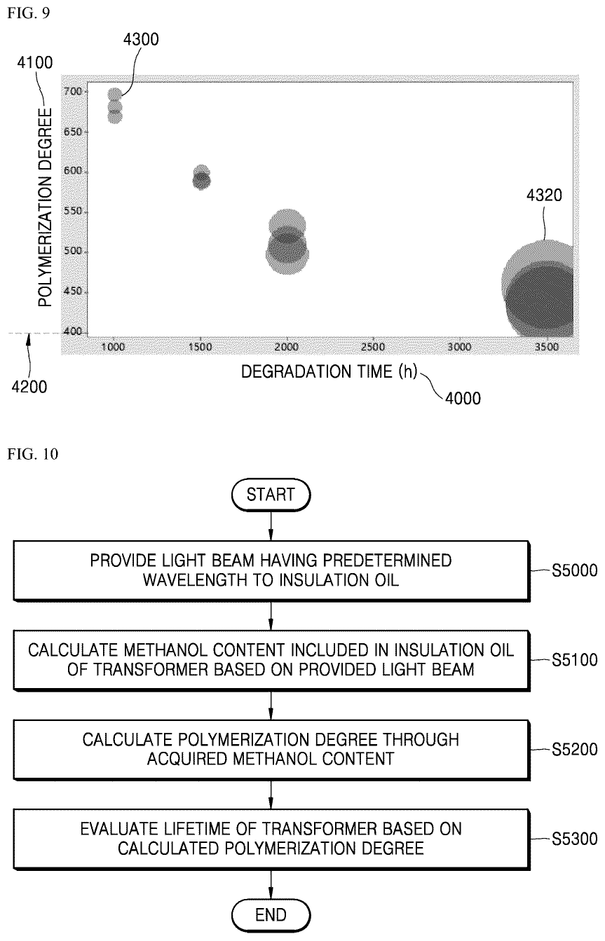

FIG. 9 is a graph depicting the polymerization degree and the change in the methanol content of the insulation oil according to the degradation time in the embodiment of the present specification.

Referring to the drawing, a horizontal axis denotes a degradation time (h) 4000, a vertical axis denotes a polymerization degree 4100, and a circle marked in the drawing denotes the methanol content included in the insulation oil. As the degradation time 4000 increases, the methanol content of the insulation oil 1620 increases due to the degradation of the insulation paper 1640, and thus a diameter of the circle increases. For example, a diameter 4320 of the circle when the degradation time is 3500 h is greater than a diameter 430 of the circle when the degradation time is 1000 h. That is, the methanol content included in the insulation oil is proportional to the degradation time 4000.

Meanwhile, the polymerization degree calculation unit 1300 can calculate the polymerization degree 4100 from the methanol content through [Equation 1].

$$DP = exp [ \frac{a - ln ( {MeOH}_{ppm} )}{b} ] \qquad [ Equation 1 ]$$

(Here, DP denotes the polymerization degree, MeOH denotes the methanol content, a denotes the first reference value, and b denotes the second reference value)

Here, the first reference value may be any value between 56.55 that is a lower limit of the first reference value and 73.5 that is an upper limit of the first reference value, and the second reference value may be any value between 8.5 that is a lower limit of the second reference value and 11.15 that is an upper limit of the second reference value.

Referring back to FIG. 9, the polymerization degree according to the methanol content, which is calculated through [Equation 1], can be identified. When the diameter 4320 of the circle is large, the polymerization degree is 650 to 700, which is greater than 400 to 500 that is the polymerization degree when the diameter 430 of the circle is small. That is, as the methanol content increases, the polymerization degree decreases.

In other words, since the polymerization degree decreases as the degradation of the insulation paper progresses, the lifetime evaluation unit 1400 may evaluate the lifetime of the insulation paper, that is, the lifetime of the transformer 1600, through the calculated polymerization degree.

In the embodiment of the present specification, the lifetime evaluation unit 1400 may preset the lifetime point limit. The lifetime point limit may be, for example, a polymerization degree of 400 that is a minimum polymerization degree at which it is determined that the transformer 1600 is unusable (indicated by reference numeral 4200). Thus, the lifetime evaluation unit 1400 may evaluate the lifetime of the transformer 1600 based on the polymerization degree of 400 and evaluate that the lifetime of the transformer 1600 is over and thus the transformer 1600 is no longer usable when the calculated polymerization degree is 400 or less.

FIG. 10 is a flowchart of the transformer lifetime evaluation method according to the embodiment of the present specification.

Referring to the drawing, the transformer lifetime evaluation apparatus 1000 provides the light beam having the predetermined specific wavelength to the insulation oil or receives the light beam having the predetermined specific wavelength (S5000). The methanol included in the insulation oil has optical properties including the absorptivity, the reflectance, and the refractive index, and the optical properties of the methanol vary depending on the content of the methanol.

Thus, the transformer lifetime evaluation apparatus 1000 calculates the methanol content included in the insulation oil of the transformer based on the provided light beam (S5100). In detail, the transformer lifetime evaluation apparatus 1000 acquires the absorbance according to the optical properties of the methanol based on the provided light beam and acquires the methanol content included in the insulation oil of the transformer based on the acquired absorbance.

Thereafter, the transformer lifetime evaluation apparatus 100 calculates the polymerization degree through the acquired methanol content (S5200). In detail, as described above, the polymerization degree can be calculated through [Equation 1] with the methanol content.

When the polymerization degree is calculated, the transformer lifetime evaluation apparatus 1000 evaluates the lifetime of the transformer based on the calculated polymerization degree (S5300). The transformer lifetime evaluation apparatus 1000 may preset the lifetime point limit and may determine that the transformer is unusable when the calculated polymerization degree is smaller than or equal to the predetermined lifetime point limit. Here, the lifetime point limit may be, for example, the polymerization degree of 400.

In this way, in the transformer lifetime evaluation method according to the embodiment of the present specification, the lifetime of the transformer can be evaluated without directly collecting the methanol through Equation 1 between the methanol included in the insulation oil and the polymerization degree of the insulation paper.

Further, in the transformer lifetime evaluation method according to the embodiment of the present specification, the lifetime of the transformer can be precisely evaluated by calculating the optical properties of the methanol through the optical sensor that uses a light beam having a specific wavelength as a light source.

Further, in the transformer lifetime evaluation method according to the embodiment of the present specification, the optical sensor that has a low cost and is easily installed and operated is used, the lifetime of the transformer can be efficiently evaluated, and thus costs can be reduced.

The present disclosure has been described above with reference to the accompanying drawings, but the present disclosure is not limited by the embodiments disclosed in the present specification and the drawings, and various modifications may be made by those skilled in the art within the scope of the technical spirit of the present disclosure. In addition, although the effects according to the configuration of the present disclosure have not been explicitly described in description of the embodiments of the present disclosure, it is apparent that the effects predictable from the corresponding configuration should also be recognized.

1. A transformer lifetime evaluation apparatus using optical properties of methanol included in insulation oil, the apparatus comprising:
a first storage part in which insulation oil used for insulating a transformer is stored;
a second storage part which is provided separately from the first storage part and in which insulation oil not used for insulating the transformer is stored;
a light emitting unit configured to emit a light beam having a specific wavelength;
a light receiving unit configured to receive the light beam emitted from the light emitting unit;
a first optical cable configured to connect the light emitting unit and the light receiving unit to provide a movement path for the light beam emitted from the light emitting unit and received by the light receiving unit and disposed to pass through the first storage part;
a second optical cable configured to connect the light emitting unit and the light receiving unit to provide a movement path for the light beam emitted from the light emitting unit and received by the light receiving unit and disposed to pass through the second storage part; and
a calculation unit connected to the light receiving unit and configured to receive information on the optical properties of the methanol from the light receiving unit.

2. The transformer lifetime evaluation apparatus of claim 1, wherein the light receiving unit transmits, to the calculation unit, the information on the optical properties of the methanol included in the insulation oil stored in the first storage part and the second storage part.

3. The transformer lifetime evaluation apparatus of claim 2, wherein the optical properties include at least one of absorptivity, reflectance, or a refractive index.

4. The transformer lifetime evaluation apparatus of claim 1, wherein the calculation unit includes:
a methanol content analysis unit configured to calculate a methanol content included in the insulation oil of the transformer based on the light beam having the specific wavelength;
a polymerization degree calculation unit configured to calculate a polymerization degree of insulation paper provided in the transformer based on the calculated methanol content; and
a lifetime evaluation unit configured to evaluate a lifetime of the transformer based on the calculated polymerization degree.

5. The transformer lifetime evaluation apparatus of claim 4, wherein the methanol content analysis unit calculates the methanol content included in the insulation oil of the transformer based on an absorbance of the methanol transmitted from the light receiving unit.

6. The transformer lifetime evaluation apparatus of claim 1, wherein the specific wavelength is in a band in a range of 317 nm to 328 nm.

7. The transformer lifetime evaluation apparatus of claim 3, wherein the calculation unit compares the optical properties of the methanol stored in the first storage part and the second storage part and determines that the transformer is unusable when a difference value between the optical properties exceeds a set range.

8. A transformer lifetime evaluation method using the transformer lifetime evaluation apparatus of claim 1, the method comprising:
emitting, by the light emitting unit, the light beam having the specific wavelength to the insulation oil stored in the first storage part and the second storage part;
transmitting, by the light receiving unit, the information on the optical properties of the methanol stored in the first storage part and the second storage part to the calculation unit; and
comparing, by the calculation unit, the optical properties of the methanol stored in the first storage part and the second storage part.

9. The transformer lifetime evaluation method of claim 8, wherein, in the comparing of, by the calculation unit, the optical properties of the methanol stored in the first storage part and the second storage part, when a difference value between the optical properties exceeds a set range, the calculation unit determines that the transformer is unusable.

10. The transformer lifetime evaluation method of claim 8, further comprising:
calculating, by the calculation unit, a content of the methanol included in the insulation oil stored in the first storage part;
calculating, by the calculation unit, a polymerization degree of insulation paper provided in the transformer based on the methanol content of the first storage part; and
evaluating, by the calculation unit, a lifetime of the transformer based on the calculated polymerization degree.

11. The transformer lifetime evaluation method of claim 10, wherein, in the evaluating of, by the calculation unit, the lifetime of the transformer based on the calculated polymerization degree,
when the calculated polymerization degree is smaller than or equal to a set lifetime point limit, it is determined that the transformer is unusable.

12. A transformer lifetime evaluation method using optical properties of methanol included in insulation oil, the method comprising:
providing a light beam having a predetermined specific wavelength to the insulation oil or receiving the light beam having the predetermined specific wavelength;
acquiring a methanol content included in the insulation oil of the transformer using the light beam having the specific wavelength;
calculating a polymerization degree through the acquired methanol content; and
evaluating a lifetime of the transformer based on the calculated polymerization degree.

13. The transformer lifetime evaluation method of claim 12, wherein the optical properties include absorptivity, reflectance, and a refractive index.

14. The transformer lifetime evaluation method of claim 12, wherein the calculating of the methanol content included in the insulation oil of the transformer based on the light beam having the specific wavelength includes:
acquiring an absorbance according to the optical properties of the methanol based on the light beam having the specific wavelength; and
calculating the methanol content included in the insulation oil of the transformer based on the acquired absorption.

15. The transformer lifetime evaluation method of claim 12, wherein the predetermined specific wavelength is in a wavelength band of a section having an absorbance of a specific magnitude or more while having a graph distribution at regular intervals.

16. The transformer lifetime evaluation method of claim 15, wherein the wavelength band of the section having the absorbance of the specific magnitude or more while having the graph distribution at regular intervals is greater than or equal to 317 nm and smaller than or equal to 328 nm.

17. The transformer lifetime evaluation method of claim 12, wherein, in the calculating of the polymerization degree through the acquired methanol content, the polymerization degree is calculated through [Equation 1],
$$DP = exp [ \frac{a - ln ( {MeOH}_{ppm} )}{b} ] \qquad [ Equation 1 ]$$
(DP denotes the polymerization degree, Me0H denotes the methanol content, a denotes a first reference value, and b denotes a second reference value).

18. The transformer lifetime evaluation method of claim 17, wherein the first reference value is any value between 56.55 that is a lower limit of the first reference value and 73.5 that is an upper limit of the first reference value, and the second reference value is any value between 8.5 that is a lower limit of the second reference value and 11.15 that is an upper limit of the second reference value.

19. The transformer lifetime evaluation method of claim 12, wherein the evaluating of the lifetime of the transformer based on the calculated polymerization degree includes determining that the transformer is unusable when the calculated polymerization degree is smaller than or equal to a predetermined lifetime point limit.

20. The transformer lifetime evaluation method of claim 19, wherein the lifetime point limit corresponds to a case in which the polymerization degree is 400.
